(19) United States
(12) Patent Application Publication
(10) Pub. No.: US 2015/0164440 A1
(43) Pub. Date: Jun. 18, 2015

(54) SETTING A RECORDING AREA

(71) Applicant: Siemens Aktiengesellschaft, Munich (DE)

(72) Inventors: Bastian RACKOW, Erlangen (DE); Martin SEDLMAIR, Zirndorf (DE)

(21) Appl. No.: 14/558,888

(22) Filed: Dec. 3, 2014

(30) Foreign Application Priority Data
Dec. 17, 2013 (DE) 102013226242.6

(51) Int. Cl. A61B 5/00; A61B 6/03; A61B 5/055; G06F 3/01

(52) U.S. Cl. CPC A61B 5/7485; G06F 3/017; A61B 5/0035; A61B 5/0077; A61B 6/03; A61B 5/055; A61B 5/0046; A61B 5/749

(57) ABSTRACT

A method is disclosed for setting a recording area of medical technology imaging via a medical technology tomography device. In an embodiment, the method includes capturing, via a number of optical and/or quasi-optical capture devices, an area of the patient table. On the basis of capture data captured thereby, an input by a user of recording area data is captured. In addition, a correspondingly embodied setting system is disclosed.

### PRIORITY STATEMENT

The present application hereby claims priority under 35 U.S.C. §119 to German patent application number DE 102013226242.6 filed Dec. 17, 2013, the entire contents of which are hereby incorporated herein by reference.

### FIELD

At least one embodiment of the present invention generally relates to a method for setting a recording area of medical technology imaging by way of a medical technology tomography device. It further generally relates to a creation system for the same purpose.

### BACKGROUND

Computed tomographs (CT), magnetic resonance tomographs (MR), angiographs, single proton emission computed tomographs (SPECT) and positron emission tomographs (PET) are typically referred to as medical technology tomography devices.

In current imaging examinations with the aid of medical tomography devices the usual sequence is as follows:

A patient or an examination object is brought into an examination room where they place themselves on a patient couch or are placed there by staff, i.e. a user of the tomography device.

Subsequently the patient is positioned at the start position by the staff with the aid of a laser marker integrated into the tomography device.

From a control room adjoining the examination room the execution of an overview recording, referred to as a topogram, is then started. With a CT recording for example, a continuous x-ray fluoroscopy recording is created by way of a fixed x-ray tube with permanent radiation.

The recording area, known as the scan range is then planned on this fluoroscopy recording, which describes the start and end position for the subsequent actual imaging scan, i.e. for the imaging. This planning is thus undertaken exclusively from the control room.

This procedure is associated with a significant outlay in time for the positioning of the start position of the topogram. The patient is mostly moved to the start position by way of the multiple back and forth movements of the patient table by way of a variety of control buttons, if necessary they have to move into a different position themselves. The end position of the scan cannot be determined at all, it is only subsequently set as a predefined position in the control room. In such cases it can also occur that a scan length that is too short is accidentally set and the topogram does not include all relevant body regions of the patient.

In addition the dose modulation of the x-ray radiation of the imaging scan is also controlled on the basis of the topogram and its attenuation information. It must thus be insured that sufficient information is present for such dose modulation, since the necessary information can otherwise only be deduced indirectly or extrapolated from the topogram or no dose modulation at all can take place locally. The latter situation means that patients themselves are unnecessarily scanned with an increased radiation dose.

One approach to resolving missing information from topogram includes carrying out a further topogram scanning and increasing the scan length of the topogram during said scan, which once again would mean subjecting the patient to unnecessarily increased radiation. As an alternative an additional topogram scan of only the missing body regions can be carried out in order to expand the originally recorded topogram. Here once again the result can however be inconsistencies in the topogram ultimately recorded (because of movements for example or even relocations of the patient), so that in this approach too there is a potential danger that the subsequent dose modulation during the imaging scan cannot be performed efficiently.

### SUMMARY

At least one embodiment of the present invention provides an option for simple and most reliable possible setting of a recording area of medical technology imaging by way of a medical technology tomography device.

A method and a setting system are disclosed.

In accordance with at least one embodiment of the invention, a method includes recording an area of a patient table via a number of optical and/or quasi-optical capture device and, on the basis of the recording data generated thereby, capturing a user entry of a user of recording area data.

According to at least one embodiment of the invention, a setting system includes a number of optical and/or quasi-optical capture device which during operation capture an area of the patient table, wherein the setting system is embodied so that, on the basis of capture data generated by the capture device, it captures a user input of recording area data. For this purpose the setting system preferably includes a recording area data derivation unit which during operation derives the recording area data from the capture data.

At least one embodiment of the invention therefore also comprises a computer program product which is able to be loaded directly into a processor of a programmable setting system, with program code segments for executing all steps of an embodiment of an inventive method when the program product is executed on the setting system.

### BRIEF DESCRIPTION OF THE DRAWINGS

The invention will be explained once again in greater detail below, referring to the enclosed figures, on the basis of example embodiments. In said explanations the same components are provided with identical reference characters. In the figures:

FIG. 1 shows a schematic block diagram to illustrate two example embodiments of the inventive method,

FIG. 2 shows a perspective view of a medical technology tomography device in accordance with a first form of embodiment of the invention,

FIG. 3 shows a front view of a display device, as can be used as part of the first example embodiment of the invention,

FIG. 4 shows a perspective view of a medical technology tomography device in accordance with a second form of embodiment of the invention,

FIG. 5 shows a schematic block diagram to illustrate example embodiments of the inventive tomography device.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

Before discussing example embodiments in more detail, it is noted that some example embodiments are described as processes or methods depicted as flowcharts. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Methods discussed below, some of which are illustrated by the flow charts, may be implemented by hardware, software, firmware, middleware, microcode, hardware description languages, or any combination thereof. When implemented in software, firmware, middleware or microcode, the program code or code segments to perform the necessary tasks will be stored in a machine or computer readable medium such as a storage medium or non-transitory computer readable medium. A processor(s) will perform the necessary tasks.

Specific structural and functional details disclosed herein are merely representative for purposes of describing example embodiments of the present invention. This invention may, however, be embodied in many alternate forms and should not be construed as limited to only the embodiments set forth herein.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

In the following description, illustrative embodiments may be described with reference to acts and symbolic representations of operations (e.g., in the form of flowcharts) that may be implemented as program modules or functional processes include routines, programs, objects, components, data structures, etc., that perform particular tasks or implement particular abstract data types and may be implemented using existing hardware at existing network elements. Such existing hardware may include one or more Central Processing Units (CPUs), digital signal processors (DSPs), application-specific-integrated-circuits, field programmable gate arrays (FPGAs) computers or the like.

Note also that the software implemented aspects of the example embodiments may be typically encoded on some form of program storage medium or implemented over some type of transmission medium. The program storage medium (e.g., non-transitory storage medium) may be magnetic (e.g., a floppy disk or a hard drive) or optical (e.g., a compact disk read only memory, or “CD ROM”), and may be read only or random access. Similarly, the transmission medium may be twisted wire pairs, coaxial cable, optical fiber, or some other suitable transmission medium known to the art. The example embodiments not limited by these aspects of any given implementation.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

In accordance with at least one embodiment of the invention, a method includes recording an area of a patient table via a number of optical and/or quasi-optical capture device and, on the basis of the recording data generated thereby, capturing a user entry of a user of recording area data.

Optical capture devices include all those devices which carry out a capture based on electromagnetic light waves, especially preferably in the visible light wave range, during operation. This includes, but is not limited to, camera and laser-based capture systems. Quasi-optical capture devices refer to all those devices which carry out a capture during operation which operate in a range other than the light wave range, on the basis of which optical images can be reconstructed. These include, but are not limited to, ultrasound capture devices. Preferably the quasi-optical capture systems are embodied such that they essentially do not emit any radiation directly damaging to human beings, i.e. x-ray or radioactive radiation.

The area of the patient table is especially defined as that area of the room which lies above the patient table, i.e. which is defined by a vertical projection of the patient table 2 above the patient table. “Above” the patient table here is the side of the patient table where the patient is also normally supported. This area, preferably also an area to the side around the patient table, is thus captured optically or quasi-optically, especially preferably at least the entire area defined by the aforementioned projection and/or the area which lies in a vertical projection above the outline of the patient. The area of the patient table can especially be delimited by the examination area of the tomography system, i.e. an inner space, such as within a gantry of the tomography system in which the patient as a rule is only located for imaging purposes.

With the aid of this optical or quasi-optical capture the location of the outline or an image of the patient can be captured and/or actions taken by a user in this area. The (quasi-) optical information generated in this way is subsequently used or is used as part of the capture process for user input.

The capture device can, for example, be connected to the tomography device or be integrated as a part thereof into the tomography device. For this purpose each capture device individually or a number of capture device together can be equipped with connection devices for connection to the tomography device. The capture device can also be arranged elsewhere, for example on a room ceiling or room wall of the examination room. If a number of capture devices are used, these can also be partly connected to the tomography device or integrated into said device, while other capture devices are arranged elsewhere.

One of the options provided by at least one embodiment of the inventive method is the setting of start and/or end position (preferably of both) of the recording area based on resilient data, namely for example the outlines of the patient and/or of markings made directly in the area of the patient table by a user.

According to at least one embodiment of the invention, a setting system includes a number of optical and/or quasi-optical capture device which during operation capture an area of the patient table, wherein the setting system is embodied so that, on the basis of capture data generated by the capture device, it captures a user input of recording area data. For this purpose the setting system preferably includes a recording area data derivation unit which during operation derives the recording area data from the capture data.

At least one embodiment of the invention also relates to a medical technology tomography device with a recording unit and with an embodiment of an inventive setting system.

Overall a large part of the components for realizing the setting system in an embodiment of an inventive manner can be realized entirely or partly in the form of software modules on a processor.

Interfaces of the setting system do not absolutely have to be embodied as hardware components but can be realized as software modules, for example when the data can be transferred from another component already realized on the same device such as an image reconstruction facility or the like for example or only has to be transferred to another component by way of software. Likewise the interfaces can consist of hardware and software components, such as for example a standard hardware interface which is configured by software for the actual intended purpose. In addition a number of interfaces can also be combined into a common interface, for example an input-output interface.

At least one embodiment of the invention therefore also comprises a computer program product which is able to be loaded directly into a processor of a programmable setting system, with program code segments for executing all steps of an embodiment of an inventive method when the program product is executed on the setting system.

Further especially advantageous embodiments and developments of the invention emerge from the dependent claims and from the description given below. In such cases the setting system can also be developed in accordance with the respective dependent claims for the method.

Preferably the user input is based on an image recorded by the capture device—in this case an optical capture device—in combination with gesture recognition. Such gesture recognition can for example be understood as the recognition of a user input on a touchscreen, wherein the recorded image is displayed on this touchscreen. With the aid of the gesture(s), for example simply touching a pushbutton but especially also an expansion of a specific selection area on the touchscreen, with said selection area representing the recording area, the desired recording area can be defined easily and especially intuitively. Further gesture recognition options are described in greater detail below.

In accordance with a first form of embodiment of the invention, the capture data is output on a display device—for example a touchscreen as explained above. The user then enters the recording area data via an input interface. The recording data is then displayed to the user via the display device, for example a monitor. In this case the display device can both be disposed in the examination room and also in the control room. A computer mouse, a joystick, a touch panel (touchscreen) but also a non-contact input interface can serve as the input interface. On the basis of the capture data displayed to them, which preferably comprises such data in particular which in the display on the display device allows feedback for the user as to the position or the outline of the patient, the user can thus input their recording area data. This input is preferably subsequently reproduced for them directly on the display device.

Within this context it is preferred that the optical capture device comprises a camera, wherein preferably with the aid of the number of optical capture device a three-dimensional (3D) depth information image is generated. Such a camera for example is a still camera (which generates still images) but preferably a video camera (which generates moving images), which thus provides a recorded image of the patient or of the area of the patient table on the basis of which the desired recording area can be defined very precisely and with a simple visual inspection. This definition in its turn can be undertaken both in the examination room and also in the control room, for example at a control monitor (especially of the tomography system). In such cases the camera preferably also operates in the visible spectrum for human beings. Preferably there is also a color image displayed based on the camera image generated by the camera which again makes navigation easier for the user.

In this case the generation of a 3D depth information image represents a preferred option with the aid of which the user is provided with a plastic image of the area of the patient table, which enables him to navigate more easily during input of the recording area data. 3D depth information can for example be generated by a single 3D camera and/or by a plurality of cameras which are arranged at different positions relative to the patient table.

According to a second form of embodiment of the invention, which can be used both as an alternative to the first form of embodiment and also to supplement said embodiment, the user input is based on a non-contact gesture recognition in the area of the patient table, wherein gestures of a user are captured by the number of optical and/or quasi-optical capture device and subsequently evaluated (by an evaluation unit), i.e. analyzed. This means that the user is located in the examination room, preferably directly beside the patient on the patient table and while he has the position of the outline of the patient precisely within view, shows the recording area. Gesture recognition accepts this display of the recording area—once again with the aforementioned optical or quasi-optical capture device—and derives the recording area data therefrom. In this case the capture device once again preferably comprises a camera, especially preferably 3D depth information as mentioned above is also generated in this context (in particular camera technology).

Preferably the gesture recognition includes a motion detection of extremities of the user. The extremities include in particular the dimensions of members, especially the arms and hands (or parts hereof, specifically the fingers) and the head of the user. Such motion detection is also referred to by the term motion tracking. Devices for motion tracking of finger movements are marketed for example under the name “Leap Motion Controller” by Leap Motion of San Francisco, USA.

It is further especially preferred that the user input takes place in the same room in which the medical technology tomography device is located, this means that the user makes his user entries where the tomography device is operating. This makes even a direct interaction between the user and the tomography device possible. Moving from this room into another room for the purposes of user control is thus not necessary and also not desired except for safety purposes, especially radiation protection.

In addition, in an advantageous manner there can be provision that by way of optical projection, for example by way of a projector or a light source (also such as a laser, for example a cone laser), a recording area currently set is shown on the patient table. Through this the user receives direct feedback during gesture recognition-based input of the recording area, as to which recording area the setting system has captured. Errors or misunderstandings in the input of the recording area can thus be directly corrected immediately and without complications.

User input by way of gesture recognition can comprise a number of logical substeps. A central substep in such cases is dimensional specification input in which the recording area data is captured. This thus involves a gesture-based length and/or width specification of the recording area in relation to patient table and/or patient, if necessary consolidated by a height specification. This dimension specification input can be done by recognition of one or more extremities, for example by displaying the start position and the end position of the scan after one another with the same hand and the same arm or simultaneously (or also consecutively) by different extremities, for example both hands or arms.

Preceding this step, but principally also as a user input alone or in combination with user inputs other than the dimensional specification input, the user input can comprise a signal input of an input initiation signal, with which a beginning of a specification of the recording area data is displayed. Such an input initiation signal thus serves to initiate the beginning of a dimension specification of the recording area—wherein the dimension specification of the recording area is not mandatorily made, but is preferably made likewise based on gestures. With the input initiation signal the setting system is switched from a standby mode into a recording mode so to speak.

Similarly to this there can be provision, after a dimension specification of the recording area, for the user input to comprise a signal input of a confirmation signal, which allows user inputs previously made and/or includes a cancellation signal which cancels previously made user inputs, especially those made temporally before an actuation signal. With the aid of the confirmation or the cancellation signal the input process of dimension specification can thus be concluded (confirmed and released for execution/further processing) or canceled once again.

This type of user input is to be seen as similar to pressing an “Enter” key or a “Delete” on the computer. It is thus insured that an incorrect input is not made by the user without the user actually wishing to do this. In addition it is possible in this way to correctly time the execution of control commands generated by the user input. As part of the invention, which can potentially be based exclusively on non-contact user entries, such a final user confirmation entry or the provision of a cancel function before the implementation of the control commands has the advantage of increased process safety and above all increasing the trust of the user in the system. This thus enables the acceptance of the user for the innovative non-contact control to be increased.

In relation to the input initiation signal and/or the confirmation signal, there is preferably provision for this to be done on the basis of a number of predefined gestures, which are captured by way of non-contact gesture recognition. This means that the signals before or after the capturing of the recording area are (also) captured gesture-based, which simplifies gesture-based capture overall.

As an alternative there can be provision for the input initiation signal and/or the confirmation signal to be based on a number of signal inputs which are captured independently of non-contact gesture recognition. In these cases a recognition logic other than the gesture recognition is used for capturing the respective signals. This potentially enables the precision or the reliability of the overall sequence of user input to be increased. For example the said signals can be issued with the aid of a touch-based input such as via a mouse click, touching a touch panel, actuating a foot switch, joystick signal and many more.

As an alternative to touch-based input of the signals, the number of signal inputs can be captured with the aid of a further non-contact user input recognition logic, especially an eye position and/or movement detection and/or a recognition of acoustic signals, especially voice signals of the user.

Use can thus be made for example of what is known as eye tracking (eye detection), a technology in which the eye position (i.e. the direction of view and/or the focusing of a human eye) and/or the movement of the eye is detected. This technology is currently used for attention research in advertising and likewise for communication with very severely disabled people. The fixing of points (fixation) in space is an intentionally controllable process, whereas eye movements (saccades) are ballistic and thus straight line and as a rule not completely intentionally controllable. Both the fixing of points and also the eye movement can currently be determined with the aid of eye-tracking and both information components can be used for recognizing a user input—the first for example as a reproduction of deliberate processes, the latter for example for verification of such a statement of intent by examining such subliminal reactions. Eye-tracking devices for computers are offered by Tobii of Danderyd, Sweden. Other eye tracking algorithms are however principally also able to be used.

Acoustic signals can comprise noises or sounds for example, as we use for example in everyday usage of speech—such as sounds for indicating yes (“mhh”) or no (“ä-äh”), especially however they comprise voice signals which can be made recognizable as user inputs with the aid of speech recognition algorithms. For example Nuance from Burlington, USA offers speech recognition software under the name Dragon Naturally Speaking which can be used within this framework. Other speech recognition algorithms are principally however able to be used within the framework of the invention.

Each of the variants has its specific advantages. Voice recognition offers the advantage that a user does not have to separately learn a “vocabulary” of eye movements in order to make user inputs, but that they can control the system completely intuitively based on their language or their sounds: Instead the speech recognition algorithm learns the vocabulary of the user. On the other hand eye movement recognition has the advantage of patients not being irritated during control of the imaging (or image reproduction) by speech information of the user or even feeling that they are being spoken to themselves.

As a development there can be provision for the user input to also include, within the framework of gesture recognition, an initiation input to initiate execution of imaging by the medical technology tomography device. After definition of the recording area by way of gesture recognition the gesture recognition can also still be used for initiating the imaging itself.

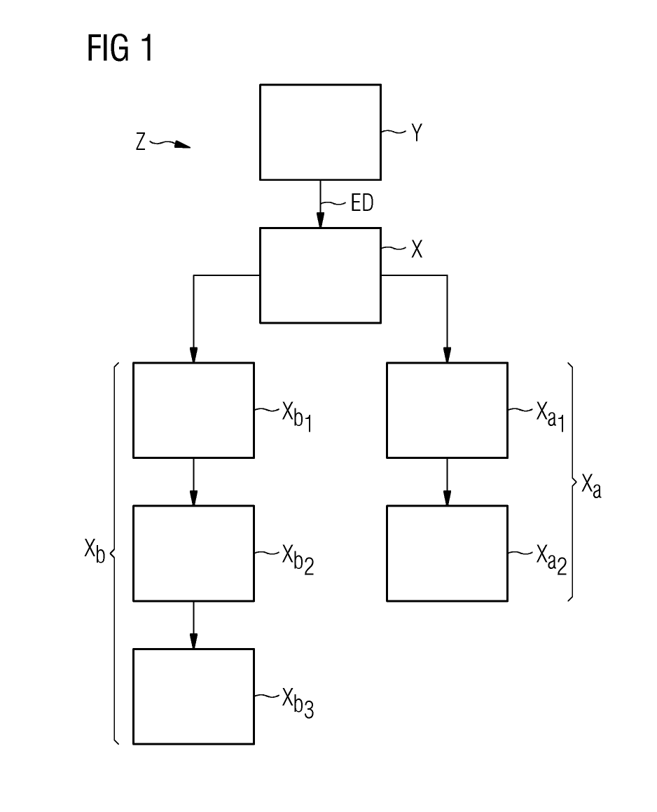

FIG. 1 shows a schematic diagram of the execution sequence of two example embodiments of the inventive method Z for setting a recording area of medical technology imaging by way of a medical technology tomography device.

In a first step Y an area of a patient table is captured by way of optical or quasi-optical capture device and capture data ED is generated therefrom. Then, in a second step X, a user input of recording area data is made by a user on the basis of the capture data.

The second step can be performed in accordance with two alternate or complementary step variants Xₐ, X_{b}.

The first step variant Xₐ provides for two substeps Xₐ₁, Xₐ₂. In the first substep Xₐ₁ in this case the capture data ED is output on a display device, for example a monitor. In the second substep Xₐ₂ a user then inputs the recording area data via an input interface.

The second step variant X_{b} is based on a recognition of gestures of the user and, in the exemplary embodiment shown here, comprises three sub-steps X_{b1}, X_{b2}, X_{b3}. The first sub-step X_{b1} comprises a gesture-based signal input X_{b1} of an input initiation signal with which a beginning of the specification of recording area data is indicated. The second sub-step X_{b2} comprises a gesture-based dimension specification input X_{b2}, in which the recording area data is captured, and the third sub-step X_{b3} comprises a likewise gesture-based signal input X_{b3} of a confirmation signal which enables the user inputs X_{b1}, X_{b2} previously made. As an alternative the third sub-step X_{b3} comprises a likewise gesture-based signal input of a cancellation signal which cancels the user inputs previously made.

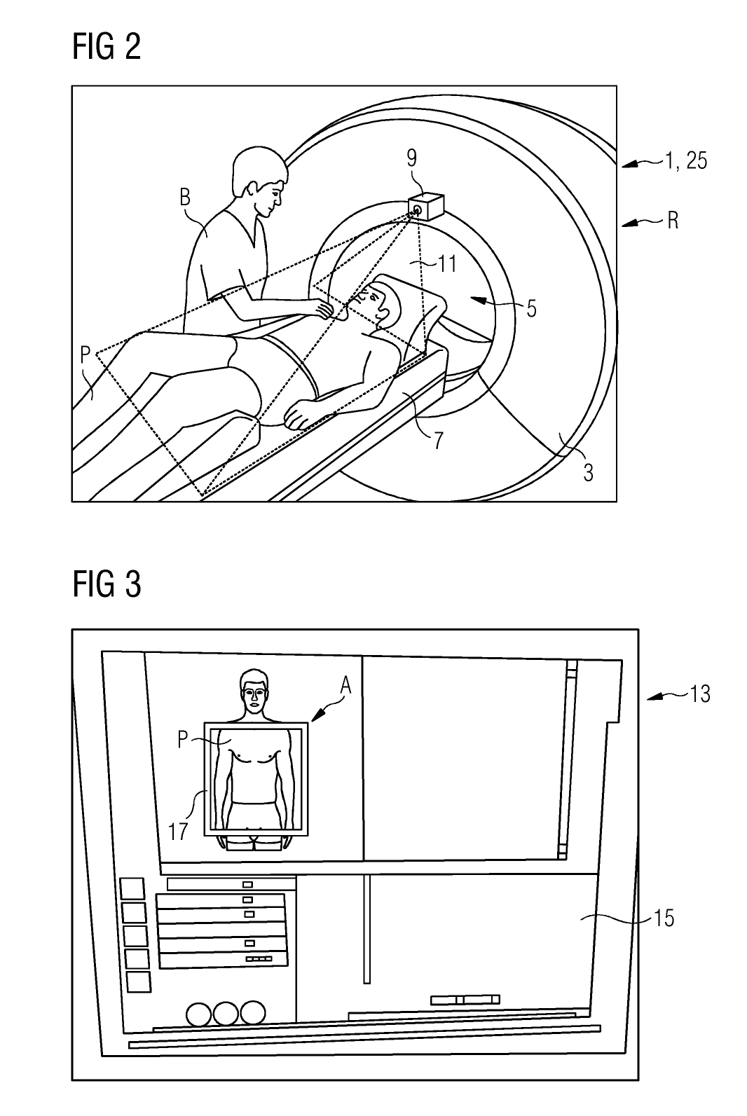

The steps of the first step variant Xₐ are explained in greater detail with reference to FIGS. 2 and 3: A medical technology tomography system 25, here a CT device 25 with a recording unit 1 which includes a gantry 3 which surrounds an examination area 5 located within the gantry 3, is located in an examination room R. A patient P is supported on a patient table 7 of the CT device 25. With the aid of a 3D camera 9 a recording area 11 is recorded in the area of the patient table 7, i.e. specifically above the patient table 7, so that the 3D camera 9 generates a 3D depth information image, for example a video image or a still image. A user B supports the patient P in accordance with specifications on the upper side of the patient table 7.

An image of the patient P on the patient table 7 is thus generated from the data captured by way of the 3D camera 9, which subsequently—see FIG. 3—is displayed to the user B on a display device 13, here a computer monitor 13. An operating menu 15 is displayed there in the lower image area of the computer monitor 13 and the image of the patient P is displayed at the top left. The recording area A is defined by the user by dragging a frame 17 around the area. This recording area A is recorded in the creation of the topogram of the patient B and/or in a subsequent CT scan (especially with dose modulation).

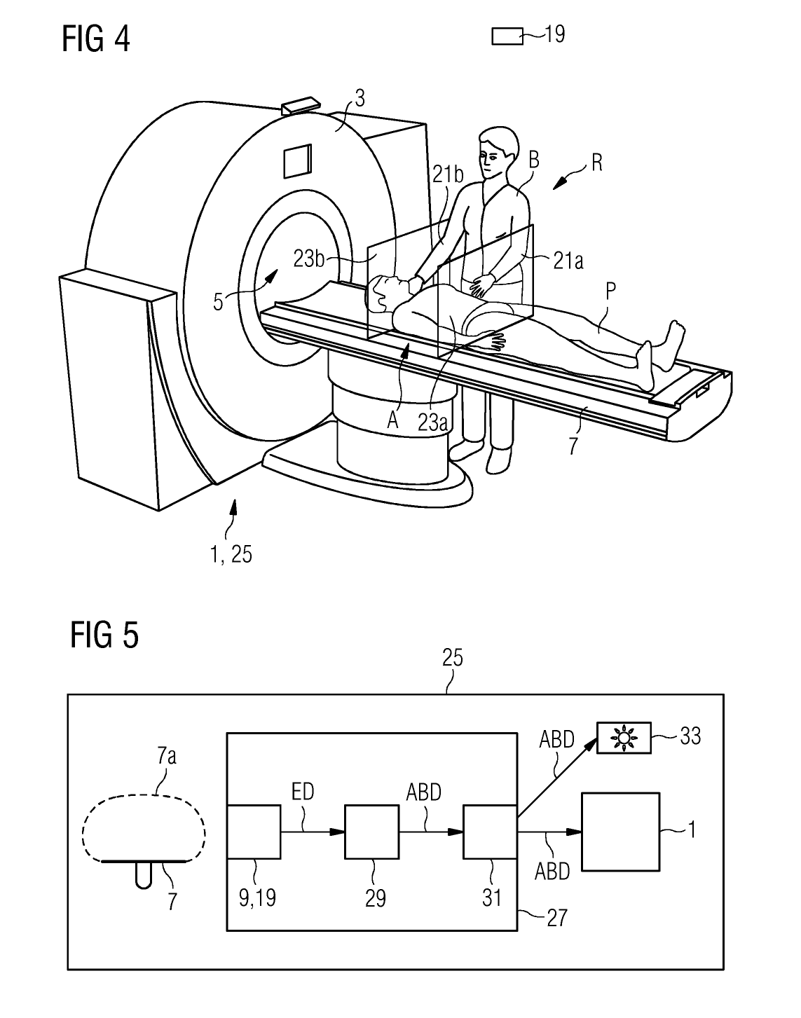

The steps of the second step variant X_{b} are explained in greater detail with reference to FIG. 4: here, similarly to FIG. 2, a patient P is supported on a patient table 7. Reference characters which are the same as those in FIG. 2 will not be explained separately again here for reasons of clarity—they relate to the same functional units as in FIG. 2. Instead of the 3D camera 9 in FIG. 2, in this step variant X_{b} a gesture recognition system 19 is used (which however usually likewise comprises a camera, namely a 3D video camera, or a number of (video) cameras, as well as a gesture recognition evaluation unit). The user B shows with his two arms 21a, 21b an extension of the desired recording area. The position of his left arm in this case corresponds to the end position of the desired recording area A, the position of his right arm to the start position of the desired recording area A.

The sequence of the gesture recognition-based step variant X_{b} can be performed as follows: The gesture recognition system 19 includes a (3D) camera which can be used to capture different height layers in the area of the patient table 7 and to correct geometrical distortions. The image information of the camera thus generated is used in a gesture recognition evaluation unit to capture gestures, for example hand and/or arm gestures of the user by way of dedicated gesture-recognition algorithms. By way of such gestures (similar to a yardstick) the user can indicate the start and end position of the recording area A and thus set said area. Here preferably only the element dimensions of the user defined previously as definitive are analyzed, especially preferably also only in the area of the patient table 7. Further gestures (for example of other limbs of the user and/or of other persons—example of the patient P—and/or outside the area of the patient table 7) are preferably not captured or are filtered/ignored in order to avoid errors in control.

The patient P is thus supported on the patient table 7. Then the user defines the recording area A by way of gestures. In order to avoid incorrect gestures a specific number of predefined gestures is preferably encoded—such as a rotation of the hands of the user B and/or the arms 21a, 21b or hands of the user B remaining in one position for a specific time.

In accordance with a development of this step variant, after the conclusion of the gesture recognition, i.e. after conclusion of the setting of the recording area A, no further resetting of the recording area is carried out, except for the user removing his arms from the area of the patient table 7 or out of the capture area of the gesture recognition system 19 and then putting them back in again.

FIG. 5 shows a schematic block diagram of an exemplary embodiment of an inventive tomography device 25 with a form of embodiment of an inventive setting system 27. The tomography device 25 also includes a patient table 7 (which can principally also be embodied independently of the tomography device 25), a recording unit 1 and an illumination unit 33 (which can principally likewise be embodied independently of the tomography device 25).

The setting system 27 comprises a number of optical and/or quasi-optical capture devices 9, 19, i.e. the 3D camera explained in greater detail above or the gesture recognition system 19 explained in greater detail above, an input and/or evaluation unit 29 and an output interface 31.

With the aid of the capture device 9, 19 and area 7a of the patient table 7 is captured. A computer mouse or another touch-based input medium and/or input medium operating in a non-contact manner can be used as input unit 29 so that the input unit 29 can make the input as part of the first step variant Xₐ. If the unit 29 is an evaluation unit 29, then it is used for example as a gesture recognition evaluation unit within the framework of step variant X_{b}. In any event the input and/or evaluation unit 29 is embodied as a generation unit 29 for generating recording area data ABD, which recording area data ABD represents the recording area A. The recording area data ABD is forwarded via the output interface 31 to the receiving unit 1 of the tomography device 25 and also optionally to the illumination unit 33. On the basis of the recording area data ABD the recording unit 1 carries out medical technology imaging in the previously defined recording area A. The illumination unit 33 can additionally indicate the defined recording area A to the user by light projection onto the area 7a of the patient table 7.

In conclusion it is pointed out once again that for the method described above in detail and also the devices shown merely involve exemplary embodiments which can be modified in a wide diversity of ways by the person skilled in the art, without departing from the field of the invention. Furthermore the use of the indefinite article “a” or “an” does not exclude the features involved also being able to be present a number of times.

The patent claims filed with the application are formulation proposals without prejudice for obtaining more extensive patent protection. The applicant reserves the right to claim even further combinations of features previously disclosed only in the description and/or drawings.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combinable features, lead to a new subject matter or to new method steps or sequences of method steps, including insofar as they concern production, testing and operating methods.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program, tangible computer readable medium and tangible computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a tangible computer readable medium and is adapted to perform any one of the aforementioned methods when run on a computer device (a device including a processor). Thus, the tangible storage medium or tangible computer readable medium, is adapted to store information and is adapted to interact with a data processing facility or computer device to execute the program of any of the above mentioned embodiments and/or to perform the method of any of the above mentioned embodiments.

The tangible computer readable medium or tangible storage medium may be a built-in medium installed inside a computer device main body or a removable tangible medium arranged so that it can be separated from the computer device main body. Examples of the built-in tangible medium include, but are not limited to, rewriteable non-volatile memories, such as ROMs and flash memories, and hard disks. Examples of the removable tangible medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for setting a recording area of medical technology imaging via a medical technology tomography device, the method comprising:
capturing an area of a patient table via a number of optical and/or quasi-optical capture devices, and generating capture data; and
capturing, on the basis of the capture data, a user input of recording area data by a user.

2. The method of claim 1, wherein the user input is made on the basis of an image recorded by the optical capture device in combination with gesture recognition.

3. The method of claim 1, wherein the capture data is output on a display device and the user inputs the recording area data via an input interface.

4. The method of claim 1, wherein the optical capture device includes a camera, and wherein a 3D depth information image is generated with the aid of a number of optical capture devices.

5. The method of claim 1, wherein the user input is made on the basis of a non-contact gesture recognition in the area of the patient table, and wherein gestures of a user are captured and evaluated by the number of optical and/or quasi-optical capture devices.

6. The method of claim 5, wherein the user input is made in the same room in which the medical technology tomography device is located.

7. The method of claim 5, wherein a currently set recording area is displayed via an optical projection onto the patient table.

8. The method of claim 5, wherein the user input includes a signal input of an input initiation signal, a beginning of a specification of the recording area data being indicated with the signal input.

9. The method of claim 5, wherein the user input includes at least one of a signal input of a confirmation signal which enables user inputs previously made and a signal input of a cancellation signal which cancels user inputs previously made, chronologically before a confirmation signal.

10. The method of claim 8, wherein at least one of the input initiation signal and the confirmation signal is made on the basis of a number of gestures which are captured by way of non-contact gesture recognition.

11. The method of claim 8, wherein at least one of the input initiation signal and the confirmation signal is made on the basis of a number of signal inputs which are captured independently of the non-contact gesture recognition.

12. The method of claim 11, wherein the number of signal inputs is captured with the aid of a further non-contact user input detection logic.

13. A setting system for setting a recording area of medical technology imaging via a medical technology tomography device, comprising:
a number of optical and/or quasi-optical detection devices, to capture an area of a patient table during operation, and generate capture data, the setting system being embodied to, on the basis of generated capture data, capture a user input of recording area data.

14. A medical technology tomography device comprising:
a recording unit; and
the setting system of claim 13.

15. A computer program product, directly loadable into a processor of a programmable setting system, including program segments to execute the method of claim 1 when the program product is executed on the setting system.

16. The method of claim 2, wherein the capture data is output on a display device and the user inputs the recording area data via an input interface.

17. The method of claim 2, wherein the optical capture device includes a camera, and wherein a 3D depth information image is generated with the aid of a number of optical capture devices.

18. The method of claim 2, wherein the user input is made on the basis of a non-contact gesture recognition in the area of the patient table, and wherein gestures of a user are captured and evaluated by the number of optical and/or quasi-optical capture devices.

19. The method of claim 12, wherein the further non-contact user input detection logic includes at least one of an eye position, eye movement detection, and a recognition of acoustic signals.

20. A computer readable medium including program segments for, when executed on a processor, causing the processor to implement the method of claim 1.
